(19) United States
(12) Patent Application Publication
(10) Pub. No.: US 2003/0087860 A1
(43) Pub. Date: May 8, 2003

(54) Methods for sexing non-human mammals

(76) Inventors: Alan Mileham, Berkeley, CA (US); Affara Nabeel, Cambridge (GB); Plastow Graham, Berkeley, CA (US)

Correspondence Address: BROBECK, PHLEGER & HARRISON, LLP, ATTN: INTELLECTUAL PROPERTY DEPARTMENT, 1333 H STREET, N.W. SUITE 800, WASHINGTON, DC, 20005, US

(21) Appl. No.: 10/137,706

(22) Filed: May 3, 2002

Related U.S. Application Data
(63) Continuation of application No. PCT/GB00/04223, filed on Nov. 3, 2001.

(51) Int. Cl. A01K 67/027; A61K 48/00

(52) U.S. Cl. 514/44; 800/8

(57) ABSTRACT

Methods for the control of sex ratio in non-human mammals are provided. These methods involve the production of transgenic animals which have particular transgenes integrated into their genomes. Animals produced using such methods are also provided, as are the transgene constructs.

This application is a continuation of Application No. PCT/GB00/04223 filed on Nov. 3, 2002, entitled “Methods for Sexing Non-Human Mammals,” and the text of the parent application is incorporated herein by reference in its entirety.

The present invention relates to methods of producing non-human animals wherein the function of sperm of a specified sex chromosome type is inhibited. Generally, this is achieved by means of introducing specific transgene constructs in order to produce transgenic animals. Suitable transgenic constructs are also provided.

The genetic sex of mammals is fixed at the moment of conception by the sex chromosome constitution of the fertilising sperm. If this sperm carries an X chromosome, the embryo develops into a female; if the sperm carries a Y chromosome, the embryo develops into a male. This, along with the fact that sperm can be easily manipulated in vitro without loosing viability, has led to many years of research into methodologies to separate X and Y chromosome bearing sperm (X and Y sperm).

The ability to produce single sex litters would be of great benefit to the agricultural industry. For example:

1. Pig producers would be able to take advantage of faster male growth rates in low slaughter weight markets or produce only females in high slaughter weight markets, thus avoiding boar taint problems and the need for castration. Single sex finishing will also make production more efficient. Slaughter plants and processors would benefit from more uniform animals. Finally the breeder would realise efficiencies in selectively producing male or female litters for boar and dam line sales. It has been estimated that the ability to produce single sex litters would be worth more than £10 million per annum in the UK alone.

2. Dairy farmers typically replace about 40% of their herd females annually with their female calves. Calves not selected to be retained in the herd (all males and 20% of the females) are sold for meat production (mainly veal). Thus semen sexing would guarantee the right number of replacement heifers to be produced while ensuring all other progeny are males sired using beef genetics.

3. In the beef and lamb industries, males are preferred as they have better growth characteristics than females. Again slaughter plants and processors would benefit from more uniform animals.

Semen sexing also has applications in humans, allowing couples at risk of producing offspring affected by sex-linked genetic disorders, to have daughters by using artificial insemination with selected X sperm. This would be preferable to the current system of amniocentesis and selective abortion to many couples.

Many claims for semen sexing systems have been made over the years based on techniques to physically separate X and Y sperm (see Hossain et al, Arch Androl 40: 3-14 (1998); Johnson, Dtsch Tierarztl Wochenschr, 103 :288-291 (1996); Windsor et al, Reprod Fertil Dev, 5 :155-171 (1993); for reviews). A number of patent applications have been filed in this area. For instance, U.S. Pat. No. 4,362,246, WO84/01265, U.S. Pat. No. 4,448,767, WO90/13303, U.S. Pat. No. 5,135,759, WO90/13315, EP-B-0 475 936, WO 91/17188, U.S. Pat. No. 4,999,283 and EP-A-0 251 710. However only fluorescence activated cell sorting (FACS) has proved to be an authentic semen sexing system. FACS involves the use of a fluorescent dye which penetrates into the nuclei of sperm cells and binds to the nuclear DNA. When isolated stained sperm are illuminated with UV light, they fluoresce and the amount of fluorescence is proportional to the amount of DNA in that sperm. Because the X chromosome is longer than the Y chromosome, sperm carrying the X chromosome contain more DNA than those carrying the Y chromosome. On this basis FACS is able to discriminate between the two sperm cell types and produce pools of separated sperm of very high purity (>90%).

Although the use of FACS separated sperm is close to commercialisation in the cattle industry, it is currently of limited use in the pig breeding industry. This is because the rate of sorting is too slow to be useful in producing sexed AI doses. Currently 3 billion sperm are required per AI dose in the pig and maximum sorting rates are in the order of 10 million per hour. This means that FACS sexed semen can only be used in pigs in combination with in vitro fertilisation and embryo transfer, neither of which are yet routine in pigs. However two groups have produced litters of pigs in this way, both in collaboration with Larry Johnson at the USDA in Beltsville USA, a pioneer of FACS technology (Rath et al, Theriogenology, 47: 795-800 (1997); Abeydeera and Day, 1998 UMC Anim Sci Dept Rep, 40-42). Both groups used IVF and surgical embryo transfer. The results of these groups showed that although some 30 embryos transferred, only about 4 survived to term.

Another concern about FACS technology is that the use of DNA binding dyes and a UV laser in the process both potentially damage sperm DNA. Although FACS practitioners claim that animals born using this technique are normal, it is highly likely that the process introduces new mutations. Conception rates following inseminations using FACS separated semen are significantly reduced which supports this view.

As FACS is able to produce highly enriched populations of X and Y chromosome bearing sperm, it has also been used as a tool in the search for more efficient semen sexing protocols. Several groups (eg Howes et al, J Reprod Fertil, 110: 195-204 (1997); Hendriksen et al, Mole Reprod Dev, 45: 342-350 (1996)) have looked for surface antigen differences between X and Y chromosome bearing sperm using 2 dimensional protein gel electrophoresis. Such differences could provide the basis of a rapid antibody based semen sorting technology. However although a patent application has been taken out for this approach (WO90/13315) none of the academic groups has been able to demonstrate reproducible differences between the surface proteins of X and Y chromosome bearing sperm. The use of ejaculated sperm may complicate these studies as sperm surfaces are extensively modified during transit through the epididymis and on exposure to seminal plasma.

Thus, there exists a continuing need to provide a reliable method for the production of semen carrying a sex chromosome of choice. We provide herein such a method, which is based on a transgenic approach. We are proposing this new approach to semen sexing, which we term Novel Semen Sexing (NSS). In this we create boars which produce either only X chromosome or only Y chromosome bearing viable sperm and thus sire either only female or only male offspring. As described above, conventional semen separation systems would never provide a practical solution to semen sexing in pigs. However, there are at least two potential problems with a transgenic approach:

1. If a boar is produced which is only capable of producing X sperm, how is such a boar reproduced without having to make new transgenic animals; and

2. Sperm cells develop in a synctium where neighbouring cells are connected by cytoplasmic bridges. This means that mRNA and proteins can be shared between X and Y sperm via these bridges. Thus, althoughsperm cells are genetically haploid, they are widely considered to be functionally diploid (see Braun et al, Nature, 337:373-376 (1989); Caldwell et al, PNAS USA, 88:2407-2411 (1991)).

Thus, in a first aspect, the present invention provides a method for the control of sex ratio in non-human mammals, which comprises the step of incorporating into the genome of said non-human mammal at least one transgene which selectively inhibits the function of those sperm having a specified sex chromosome type.

A transgene is used which inactivates sperm function. This can be achieved in several ways, for example, the transgene can comprise a sequence coding for an antisense molecule which interferes with the normal expression of sperm function, or can code for expression of an enzyme (eg RNase) which prevents the normal expression of sperm function. The transgene can be inserted into either the X or Y chromosome, thus allowing for the production of transgenic males capable of producing only male or only female offspring.

Suitably, the expression of the transgene is restricted to post-meiotic spermatids by the use of an appropriate promoter that is only expressed in such cells, for example the promoter for the protamine 1 gene, or the testis specific promoter within the sixteenth intron of the cKIT gene (Albanesi, et al, Development, 122:1291-1302 91996)). This will guarantee that the transgene only expresses in the right cells in the testis and nowhere else.

In a preferred embodiment, the expression of the transgene is further controlled by a site-specific recombinational switch such as the cre/lox system (see Sauer 1998, Methods 14, 381-392), or the FLP/FRT system (see Dymecki and Tomasiewicz 1998, Dev Biol 201, 57-65), both of which have been used to control transgene expression in transgenic mice. Other possible recombinational switches might include the Gin system from bacteriophage Mu, which has been used to promote site-specific recombination in plant protoplasts (Maeser and Kahmann 1991, Mol Gen Genet 230, 170-176), as well as modifications of inversion-mediating systems such as the Hin system of Salmonella (see Johnson and Simon 1985, Cell 41 781-791). Any site-specific recombination system capable of working in mammalian cells would serve this purpose

Furthermore the expression of the site specific recombination system itself could be controlled by the use of a promoter that was activated by an external agent. In this way the ultimate expression of the sperm inactivating transgene would be controlled by application of an external agent at a selected time. This would mean that transgenic males would produce normal sperm until the external agent was applied and in this way allow normal breeding from transgenic males and ensure their replacement even if the transgene was inserted into the Y chromosome. Examples of such controllable promoters include those from the tetracycline-inducible system (see Forster et al 1999, Nucleic Acids Res 27 708-710), the ecdysone gene (see No et al 1996, Proc Natl Acad Sci USA 93 3346-3351), the RU486-indcuible system (see Wang et al 1997, Nature Biotechnol 15 239-243), the zinc-induced metallothionine gene (see Suppola et al 1999, Biochem J 338 311-316) and the CYP1A1 gene (see Campbell et al 1996, J Cell Sci 109 2619-2625). Any promoter that can be induced by an exogenous agent in mammalian cells would serve this purpose.

In addition, the action of the transgene can be directed to a specific cell compartment such as the nucleus, acrosome or flagellum. It is expected that this approach combining late post-meiotic transgene expression with targeting the transgene product to a specific cell compartment will overcome the syncitium issue. This targeting could be achieved in several ways. For example the action of antisense RNA is thought to be within the nucleus; proteins such as RNase can be directed to the nucleus using a nuclear localisation sequence. This nuclear involvement makes it very unlikely that the sperm inactivating effect of the transgene will pass between neighbouring cells in the syncitium via the cytoplasmic bridges.

The offspring of transgenic boars, produced as described above, carrying an activated transgene, never inherit the transgene. Thus the transgene does not enter the food chain.

Thus this system delivers:

1. A transgenic (NSS) boar which produces normal ratios of X and Y sperm (and thus is able to reproduce the next generation of transgenic boars by normal breeding) until the transgene is activated by a combination of an externally activated promoter and a recombinational switch. The recombinational element of the switch means that the controllable promoter need only be activated one time.

2. Once the switch is activated, sperm derived from spermatids carrying the transgene inserted into a sex chromosome will become inactivated and thus unable to fertilise oocytes. In this way the NSS boar will produce only viable sperm of one sex chromosome constitution and thus produce either only male or only female offspring depending on whether the transgene is carried on the X or the Y chromosome.

3. Since sperm carrying the transgene are inactivated, the offspring of activated transgenic boars are not themselves transgenic, thus provided that the NSS boar lines themselves are clearly identified and incinerated after death, the transgene never enters the human food chain.

NSS Components

1. Late Spermatogenesis Promoter (P_{LATE}).

This is required to ensure that transgene expression is limited to male germ cells after meiosis. Expression in other tissues is likely to be deleterious and expression prior to meiosis in germ cells will lead to sterility. The promoter of any gene which is uniquely transcribed in post-meiotic male germ cells could be used here for example protamine 1 (Prm1), or the testis specific promoter within the sixteenth intron of the cKIT gene. It is likely that best results will be obtained with promoters that express very late in post-meiotic germ cells. The syncitial bridges will be breaking down at this stage and so present less of a barrier to this approach.

2. Stop Expression Sequence Flanked by sites for a site specific recombinase. Expression of the Sperm Function Inhibitor transgene is initially prevented using a stop expression sequence, for example a polyadenylation signal. This allows normal breeding from transgenic NSS boars until they are required to produce only one sex of offspring. The stop expression sequence is flanked by recombinational sites (such as lox P sites) which provide sites for a site specific recombinase (such as the cre recombinase). Expression of the recombinase catalyses the deletion of the stop expression sequence and thus allows the expression of the Sperm Function Inhibitor transgene in post-meiotic male germ cells.

3. Sperm Function Inhibitor (SFI).

This comprises the transgene coding or antisense regions. This must interfere with sperm function and disable any cells which express the transgene. Ideally the SFI would only have an effect in sperm cells so that any deleterious consequence of inappropriate transgene expression in other tissues is minimised. Candidates include antisense or ribozyme strategies involving essential sperm functions such as metabolism, egg recognition and binding, or motility. Other approaches include the expression of a protein which abolishes sperm function such as a general Rnase (expression here would destroy all mRNA within the sperm cell and hence all sperm function), or a surface antigen (expression here would produce antigenically distinct X and Y sperm and thus allow sperm sexing on this basis).

4. Nuclear Localisation Sequence Gene expression in post-meiotic male germ cells is complicated by the fact that the four spermatids which derive from a single spermatocyte remain connected via cytoplasmic bridges. The connected cells are termed a syncitium. This means that haploid germ cells can be thought of as functionally diploid, as mRNA or proteins derived from genes expressing post-meiotically can pass between individual cells of the syncitium via the cytoplasmic bridges. Potentially this could make the NSS approach null and void. However if a sequence is added to the 5′ end of the SFI transgene which directs the protein encoded to a particular cell compartment such as the cell nucleus (nuclear localisation sequence), acrosome or flagellum, then it is highly likely that the mRNA and protein will remain within the cell which carries that gene.

5. Externally Controllable Promoter (P_{EC}).

This will allow the expression of the site specific recombinase (eg cre) to be precisely controlled by the application of a specific inducer. Ideally this should be a promoter/inducer combination which is not found in mammals to minimise the chances of inappropriate or accidental induction of cre recombinase expression. Examples of controllable promoters include those from the tetracycline-inducible system, the ecdysone gene, the RU486-inducible system, the metallothionine gene and the CYP1A1 gene.

6. The Site Specific Recombinase.

Expression of the site specific recombinase (eg cre) will result in the deletion of the stop expression sequence via the recombinagenic sites (eg loxP) and thus the expression of the SFI transgene in post-meiotic male germ cells. Alternative site specific recombination systems include the FLP/FRT system, the Gin system from bacteriophage Mu, or an inversion-mediating system such as the Hin system of Salmonella.

7. X or Y Specific Sequence to Target Transgene.

If the transgene is targeted to the Y chromosome then after induction of the site specific recombinase, all sperm cells which carry the Y chromosome will be infertile. Here transgenic males would only be able to father daughters. Equally males carrying the transgene on the X chromosome would only father sons following induction of the site specific recombinase. Targeting may be achieved by flanking the transgene with several thousand base pairs of DNA from the target chromosome. This promotes homologous recombination between the transgenic construct and the target chromosome in embryonic stem cells. Homologous recombination requires 100% identity in DNA sequence between the transgenic construct and target chromosome. This means that it is essential that the source of target chromosome DNA in the transgenic construct comes from the embryonic stem cell line. For our purposes here we will integrate the NSS transgenic construct into the X chromosome to demonstrate the system.

If porcine embryonic stem cells are not available, NSS can still be achieved using a gene targeting strategy and nuclear transfer or by conventional transgenesis and screening for insertions into the X or Y chromosome.

A simpler system is possible if animals producing only male offspring are desired. Here the transgene simply consists of a promoter which is only expressed in post-meiotic male germ cells, driving the SFI transgene (as described above). This is targeted to the X chromosome using several thousand base pairs of DNA from the X chromosome and homologous recombination. Transgenic males here express the transgene as soon as spermatogenesis begins and only ever produce fertile sperm carrying the Y chromosome. Thus, only male offspring are produced. Females carrying a single copy of the transgene on one of their X chromosomes can then be used to generate new transgenic males by normal breeding. On average 50% of the male offspring of such carrier females will be transgenic NSS boars.

In this simple system it is important to bear in mind that female embryonic stem cells, or totipotent tissue culture cells must be used in the gene targeting. If male cells are used, the transgene will express in the chimaeric or transgenic offspring and so only fertile sperm carrying the Y chromosome will be produced. Thus, the transgene will never be inherited and a transgenic line cannot be established. However, if female cells are used in targeting, transgenic females carrying the transgene on one of their X chromosomes can be produced. When these females are bred to non-transgenic males, half of her male offspring will be NSS males and half of her female offspring will be carrier females. This allows for the continuous production of NSS males through the establishment of carrier female lines.

The invention will now be described with reference to the following examples, which should in no way be construed as limiting the scope of the invention.

### EXAMPLE 1

1. Demonstration that the Syncitial Bridge Problem can be Overcome

Systems to show that it is possible to ensure that the action of the SFI only occurs in spermatids where the transgene is inserted into one of the sex chromosomes, were developed to demonstrate that the potential problem of the syncitial bridges can be overcome. Two methods were tested; the use of a nuclear localisation sequence (nls) and anti-sense.

a) nls Approach

A construct was made fusing the mouse protamine 1 promoter (see Zambrowicz and Palmiter 1994 Biol Reprod 50, 65-72) with the nls-lacZ gene from pSKT (Stratagene). Male transgenic mice carrying this construct in a single location on one chromosome (hemizygous), would be expected to express the lac Z gene in the spermatids of their testes. This could be revealed by staining testis sections histochemically with X-Gal (see Ave et al 1997 Transgenic Res 6, 37-40).

If the nls functions as expected and directs β galactosidase to the nucleus of the cell expressing the lacZ gene, then only 50% of the spermatids will stain blue, demonstrating that the syncitial bridge problem can be overcome in this way.

Other constructs will also be made to test the ability of the nls sequence to direct transgenic gene products to the spermatid nucleus and thus avoid the syncitial bridge limitation. For example using genes encoding green or yellow fluoresence proteins as reporters, or the testis specific promoter within the sixteenth intron of the cKIT gene.

b) Antisense Approach

This approach involves the use of two transgenes. The first is the same as in Example 1a (sense); the second is a fusion between the protamine 1 promoter and an inverted lacZ gene (antisense). The latter construct would produce an antisense lacZ when expressed. The expression from transgenes varies according to their site of integration. Here several transgenic mouse lines will be made for each transgene and the level of transgene expression in the testis determined using either Northern hybridisation or RT-PCR, and a reference gene such as β actin. In this way we would identify sense and antisense lines where the expression of the sense construct was at least 10 fold lower than the antisense. However lacZ expression from the sense construct would still have to be detectable histochemically. The sense transgenic line will be bred to homozygosity and then bred with the hemizygous antisense line.

If the antisense approach works as expected by inhibiting the expression of the sense construct within the same nucleus, then histochemical staining of the testes of males from this cross would reveal only 25% of the spermatids as staining blue. Again this would demonstrate that the syncitial bridge problem could be overcome. If the approach does not inhibit sense gene expression, 50% of the spermatids would stain blue and if the approach does inhibit sense gene expression but fails to overcome the syncitial bridge problem then 0% of the spermatids would stain blue.

Alternative constructs are being made using genes encoding green or yellow fluoresence proteins as reporters, or the testis specific promoter within the sixteenth intron of the cKIT gene.

c) Flagellum Targeting

We have constructed a transgene comprising the testis specific promoter within the sixteenth intron of the cKIT gene driving a green fluorescence protein-tubulin gene fusion. We expect this fusion protein to be directed to the flagellum in elongating spermatids. If this approach works then 50% of the elongating spermatids in the testes of transgenic mice carrying such a transgene will fluoresce. However, if all spermatids show fluorescent flagella, then it wil be clear that this approach does not overcome the syncitial bridge problem, at least with the promoter used.

2. Demonstration of the NSS Principle

This example involves proof of principle of the NSS concept using a simple system. The full NSS concept relies on a recombinational switch as we require males that produce both Y chromosome only and X chromosome only sperm, yet are capable of maintaining the line through normal breeding. If we only target the X chromosome, then not only can we produce NSS males that only produce viable Y chromosome sperm, but we can also maintain the transgenic line and produce new NSS males through carrier females.

Several constructs are being made for this proof of principle. These involve the use of alternative X chromosome targeting sequences and SFIs.

a) Regions of non-essential genes expressed on the X chromosome were required for targeting the transgene to the mouse X chromosome. The Smcx (see Agulnik et al 1999 Mamm Genome 10, 926-929) and Hprt (see Konecki et al 1982 Nucleic Acids Res 10, 6763-6775; Hatada et al 1999 j Biol Chem 274, 948-955) genes were selected for this purpose. Approximately 5 kb regions of each gene were cloned from mouse strain 129/5 vEv.

b) Two approaches to the SFI have been taken; the use of proteins targeted to the nucleus using an nls, and the use of antisense. The proteins chosen were Barnase, a general Rnase which would destroy all gene expression within the nucleus and thus halt spermatid maturation (see Goldman et al 1994 EMBO J 13, 2976-2984), and HSV tk gene which is known to disrupt sperm development if expressed in post-meiotic germ cells (see Braun et al 1990 Biol Reprod 43, 684-693).

For the antisense, segments of the following genes were selected to make antisense constructs consisting of single genes, or fusions of two, three, or four genes; Sperm adhesion molecule (Spam1, see Zheng and Martin-Deleon 1997 Mol Reprod Dev 46, 252-257), fertilin beta (Ftnb, see Cho et al 1997 Dev Genet 20, 320-328), the testis-specific glyceraldehyde 3-phosphate dehydrogenase (GAPD-S, see Welch et al 1992 Biol Reprod 46, 869-878) and the testis-specific glucose 6 phosphate dehydrogenase (G6PDH, see Erickson 1975 Biochem Biophys Res Commun 63, 1000-1004). All of these mouse genes are only expressed in post-meiotic cells in the testis and so are ideal targets to disrupt for the NSS approach.

All transgenes for either the nls or antisense approach would be fused to and expressed from the mouse protamine 1 promoter.

Transgenes consisting of the protamine promoter or the testis specific promoter within the sixteenth intron of the cKIT gene, driving an appropriate SFI, embedded within an appropriate X chromosome targeting sequence would be fused to appropriate selectable markers and inserted into the X chromosome of an appropriate mouse embryonic stem cell line (see Bronson and Smithies 1994 J Biol Chem 269, 155-158). Authentic transgenic cells carrying the transgene on the X chromosome would then be injected into the blastocoel of blastocysts from appropriate mouse strain and re-implanted into the uteri of appropriate pseudopregnant recipient mice. Germline chimaeric mice would be identified using methods to detect the presence of the transgene (eg PCR or Southern hybridisation) and bred to produce trangenic lines.

Female mice would be expected to breed normally with non-transgenic mice to maintain the transgenic lines. However male transgenics would be expected to produce only non-transgenic males when bred to non-transgenic females. This result would provide proof of principle of the NSS concept.

1. A method for the control of sex ratio in non-human mammals, which comprises the step of incorporating into the genome of said non-human mammal at least one transgene which selectively inhibits the function of those sperm having a specified sex chromosome type.

2. A method as claimed in claim 1 wherein the transgene comprises a sequence coding for a protein which, when expressed, prevents the normal function of the sperm.

3. A method as claimed in claim 2 wherein the sequence codes for an RNase or thymidine kinase.

4. A method as claimed in claim 1 wherein the transgene comprises a sequence which when transcribed produces an RNA molecule which prevents the normal function of the sperm.

5. A method as claimed in claim 4 wherein the RNA molecule is an antisense molecule.

6. A method as claimed in claim 5 wherein the antisense RNA molecule binds to mRNA, which is present in post-meiotic male germ cells and which is transcribed from one or more genes critical for sperm function.

7. A method as claimed in claim 6 wherein the one or more genes is/are selected from fertilin B, sperm adhesion molecule (spam-1), glyceraldehyde phosphate dehydrogenase (GAPDH) and glucose-6-phosphate dehydrogenase.

8. A method as claimed in claim 1 wherein the transgene is under the control of regulatory sequences such that the transgene functions after meiosis in male germ cells.

9. A method as claimed in claim 8 wherein the regulatory sequence is the promoter from the protamine 1 gene or the testis specific promoter within the sixteenth intron of the cKIT gene.

10. A method as claimed in claim 1 wherein expression of the transgene is prevented in pre-activated males by the presence of a stop expression sequence.

11. A method as claimed in claim 10 wherein said stop expression sequence is a polyadenylation signal.

12. A method as claimed in claim 10 or claim 11 wherein said stop expression sequence is flanked by flanking sequences for a site specific recombination system.

13. A method as claimed in claim 12 wherein said flanking sequences are loxP or FRT sites, or sequences for site specific recombination systems from bacteriophages lambda or mu, or from bacteria.

14. A method as claimed in claim 12 wherein said stop expression sequence can be deleted following expression of a site specific recombinase, which acts upon said flanking sequences flanking the stop expression sites.

15. A method as claimed in claim 14 wherein the site specific recombinase is cre, FLP, or from site specific recombination systems from bacteriophages lambda or mu, or from bacteria.

16. A method as claimed in claim 14 wherein expression of the site specific recombinase is under the control of regulatory sequences which are controllable, such that expression can be activated when desired.

17. A method as claimed in claim 16 wherein expression is induced following the application of a specific inducer to the animal, for example in its feed or by intravenous injection.

18. A method as claimed in claim 16 wherein the regulatory sequences are provided by a promoter which is the promoter from the CYP1 A1 or CYP 2B1 gene, and induction is achieved using PAH, TCDD, beta NF, PCBs or 3-mc.

19. A method as claimed in claim 1 wherein the product of the transgene is directed to the cell nucleus by the use of a nuclear localisation sequence.

20. A method as claimed in claim 1 wherein the product of the transgene is directed to the acrosome or flagellum.

21. A method as claimed in claim 1 wherein the transgene is integrated into either the X or Y chromosome by means of sequences from expressed, but non-essential, regions of the X or Y chromosome respectively in the target species.

22. A method as claimed in claim 1 wherein the transgene is integrated into the genome of embryonic stem cells.

23. A method as claimed in claim 22 wherein selected transgenic embryonic stem cell lines are injected into morulae or blastocysts, and embryos thus manipulated are transferred to suitable recipients.

24. A method as claimed in claim 23 which results in chimaeric animals which are capable of transmitting the transgene in their germ line.

25. A method as claimed in claim 1 wherein the transgene is integrated into the genome of totipotent cells in tissue culture.

26. A method as claimed in claim 25 wherein selected transgenic totipotent cells are used as nuclear donors in a nuclear transfer procedure.

27. A method as claimed in claim 26 wherein transgenic reconstructed embryos are transferred to suitable recipients.

28. A method as claimed in claim 1 wherein the transgene is integrated into one of the sex chromosomes of fertilised eggs following pronuclear injection, lipofection, electroporation or transfection.

29. A method as claimed in claim 28 wherein manipulated embryos are transferred to suitable recipients.

30. A method as claimed in claim 29 which results in chimaeric animals which are capable of transmitting the transgene in their germ line.

31. A method as claimed in claim 1 wherein the non-human mammal is a pig, cow, sheep, goat, rabbit or mouse.

32. A non-human mammal produced according to a method as claimed in claim 1.

33. Progeny of a non-human mammal as claimed in claim 32.

34. A transgene construct suitable for use in incorporating a transgene into the genome of a non-human animal by the method as defined in claim 1.
